(19) United States
(12) Patent Application Publication
(10) Pub. No.: US 2026/0134965 A1
(43) Pub. Date: May 14, 2026

(54) PORTAL AND METHOD TO ENABLE A COMPLIANT COMMUNICATION PROTOCOL OVER A DISTRIBUTED NETWORK FOR THE E-TRANSFER OF PRESCRIPTIONS BETWEEN PHARMACIES

(71) Applicant: Four Leaf Rx, LLC d/b/a TransferMyRx, Plano, TX (US)

(72) Inventors: Nabil Adnan Hallak, Frisco, TX (US); Michael Lee Sands, Dallas, TX (US); Stephen Lanier Sands, Plano, TX (US); Mahdi Al Hallaq, Murphy, TX (US)

(73) Assignee: Four Leaf Rx, LLC d/b/a TransferMyRx, Plano, TX (US)

(21) Appl. No.: 19/442,451

(22) Filed: Jan. 7, 2026

Related U.S. Application Data
(63) Continuation-in-part of application No. 18/209,911, filed on Jun. 14, 2023.
(60) Provisional application No. 63/352,493, filed on Jun. 15, 2022.

(51) Int. Cl. G16H 20/10; G06F 9/54

(52) U.S. Cl. CPC G16H 20/10; G06F 9/54

(57) ABSTRACT

A method for enabling electronic prescription transfers between unaffiliated pharmacies operating on different pharmacy management systems (PMS) comprises receiving a prescription transfer request from a first pharmacy through either an integrated PMS interface or a secure web-based portal; routing the request through a host system; translating the request into a communication format compatible with a receiving PMS; and delivering the translated prescription to the receiving pharmacy, wherein the prescription is ingested directly into the receiving PMS when the receiving PMS is integrated with the host system or presented through the web-based portal when the receiving PMS is not integrated. The method enables compliant prescription transfers even when only one of the participating pharmacies is integrated with the host system.

### CROSS-REFERENCE TO RELATED APPLICATION(S)

The present application is a Continuation-in-Part application of U.S. patent application Ser. No. 18/209,911, entitled PORTAL AND METHOD TO ENABLE A COMPLIANT COMMUNICATION PROTOCOL OVER A DISTRIBUTED NETWORK FOR THE E-TRANSFER OF PRESCRIPTIONS BETWEEN PHARMACIES, which claims priority to U.S. Provisional Ser. No. 63/352,493 , filed on Jun. 15, 2022, the entirety of which are incorporated by reference herein.

### BACKGROUND

The present invention relates to compliant communication protocols over a distributed network and more particularly to compliant communication protocols over a distributed network between pharmacies.

Prescription control and accuracy are critical to the pharmacy industry. Advances in the industry have been relatively stagnant and there is a large unsolved need in the industry for technological developments that would enable pharmacists to do their job more efficiently and without compromising prescription accuracy and/or bypassing controls set in place to protect patients.

Presently, customers must be located near their designated pharmacy in order to obtain a prescription prescribed by their health care provider. This often becomes a tedious task for customers who may not be located near their pharmacy when medication is needed (such as when traveling) or who simply want to use a different pharmacy. In order to have the prescription order transferred to a different nearby pharmacy, customers must either go into the pharmacy physically or contact their pharmacy telephonically to request that the pharmacist call the customer's designated pharmacy to request the transfer of the customer's prescription. However, this process is antiquated, inefficient and problematic as it places the burden on the respective pharmacies to connect with each other in a timely, efficient, and effective manner. For example, it may require the schedules of a pharmacist in the transferring pharmacy and a pharmacist in the receiving pharmacy to coincide between tasks and during a short window (e.g. a few hours)-so that both can direct time and attention to verifying a communication to convey the required information used to transfer the prescription. And regulations require that the transfer be performed by pharmacists. As such, there is a need to more easily, quickly, and efficiently streamline the process for pharmacies to transfer customer prescription orders between one another to provide more widespread access to customers in need of prescribed medication.

Moreover, known pharmacy management software (PMS) system may comprise one or more modules configured to enable the intake, processing, dispensing, and regulatory management of prescriptions. Conventional PMS systems are generally designed to operate within a single corporate or vendor-controlled ecosystem, where interoperability is limited to pharmacies that share a common software infrastructure. Such systems cannot natively support the secure, compliant transfer of prescriptions between unaffiliated pharmacies operating on different PMS platforms. As such, there is further need for a system that enables the interoperability and communication between unaffiliated PMS systems that utilize and accept different communication formats.

### SUMMARY OF THE INVENTION

Embodiments of the present invention address deficiencies of the art in respect to compliant communication protocol over a distributed network between pharmacies and provide a novel and non-obvious method, system or computer program product to enable a compliant communication protocol over a distributed network for the E-transfer of a prescription between unaffiliated pharmacies that do not share the same data.

According to one or more embodiments, a method for enabling the interoperability of two or more unaffiliated pharmacies operating on separate pharmacy management (PMS) systems comprises: receiving, by a Host System, a prescription transfer request submitted by a user associated with a transferring pharmacy through a web-based Transfer Portal, wherein the transferring pharmacy does not use a PMS integrated with the Host System; identifying, by the Host System, a communication format of the prescription transfer request; translating, by a prescription translation module of the Host System, the prescription transfer request into a communication format compatible with a receiving PMS system used by a receiving pharmacy that is integrated with the Host System; transmitting the translated prescription transfer request to the receiving PMS system through an API Layer; and ingesting, by the receiving PMS system, the translated prescription transfer request into its native prescription processing workflow such that the receiving PMS system processes the prescription transfer request as if the transferring pharmacy were integrated with the Host System.

In one aspect, the format compatible with the receiving PMS system is at least one of XML, JSON, and HL7-formatted healthcare messages encoded in one of XML or JSON.

In another aspect, translating the prescription transfer request comprises mapping and converting individual data elements from the format of the first PMS system to the format compatible with the receiving PMS system.

In another aspect, translating the prescription transfer request further comprises parsing information associated with a pharmaceutical prescription and validating the presence of at least one critical element.

In another aspect, the at least one critical element comprises a patient identifier, a prescriber identifier, and a pharmaceutical drug identifier.

In another aspect, the transferring PMS system and receiving PMS system are in communication via an intermediary Host System, the Host System comprising an API Layer and web-based Transfer Portal.

In another aspect, the Host System further comprises a database storing a registry of one or more member pharmacies that are integrated with Host System.

In another aspect, the Host System comprises a prescription translation module, the prescription translation module has: format detection logic; a data parsing and validation engine; a data mapping and transformation engine; and output logic.

In another aspect, the step of translating the prescription transfer request to a second communication formation comprises: identifying the first communication format of the prescription transfer request from the transferring PMS system; parsing data element associated with the prescription transfer request; converting the data elements into a canonical data model (CDM); determining which communication formats are compatible with the receiving PMS system; and mapping the data elements into the second communication format that is compatible with the receiving PMS system.

In another aspect, the prescription translation module further comprises an ingestion gateway programmed to accept data through one or more ingress channels.

According to one or more other embodiments, a prescription transfer system comprises a Host System comprising an API Layer and a prescription translation module. A web-based Transfer Portal is in communication with the Host System. The system further comprises a transferring PMS system associated with a pharmacy sending a prescription transfer request; a receiving PMS system associated with a pharmacy receiving the prescription transfer request. One of the transferring PMS system and receiving PMS system is integrated with the Host System and the other is not. The Host System translates and delivers the prescription transfer request: to the receiving PMS via the API Layer when the receiving PMS is integrated; and for presentation to a user associated with the receiving PMS via the Transfer Portal. The Transfer Portal facilitates communication between the transferring PMS system and the receiving PMS system.

In one aspect, the Host System is configured to receive a prescription transfer request from the transferring PMS system and identify the communication format of the prescription transfer request.

In another aspect, the Host System is further configured to identify a communication format compatible with the receiving PMS system and translate the prescription transfer request to a communication format compatible with the receiving PMS system.

In another aspect, the prescription translation module comprises format detection logic programmed to identify a communication protocol of an incoming information payload; a data parsing and validation engine; a data mapping and transformation engine; and output transformation logic.

In another aspect, the prescription translation module comprises a data parsing and validation engine programmed to parse data elements associated with the prescription transfer request.

In another aspect, the prescription translation module comprises a data mapping and transformation engine programmed to convert data elements from a communication format of the prescription transfer request to a standardized canonical data model format.

In another aspect, the prescription translation module comprises output transformation logic that maps the data elements into the standardized canonical data model format to a format compatible with the receiving PMS system.

According to one or more embodiments, a prescription transfer system comprises a Host System comprising an API Layer and a prescription translation module. A web-based Transfer Portal is in communication with the Host System. The system further comprises a transferring PMS system that is not integrated with the Host System and a receiving PMS system integrated with the Host System. The Transfer Portal provides a web-based interface for the transferring PMS system to submit prescription transfer requests to the Host System, and the API Layer delivers the translated prescription data for direct ingestion into the receiving PMS system.

In one aspect, the prescription translation module and API Layer are configured such that the receiving PMS system ingests and processes the prescription transfer request as if the transferring PMS system were natively integrated with the Host System, even though the transferring PMS system is not integrated.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 shows an exemplary prescription order transfer system, in accordance with the principles of the present application;

FIG. 2 shows an exemplary computing device of the system of FIG. 1, in accordance with the principles of the present application;

FIG. 3 shows an exemplary server of the system of FIG. 1, in accordance with the principles of the present application;

FIG. 4 shows an exemplary “Transfer In” request form generated by the system of FIG. 1, in accordance with the principles of the present application;

FIG. 5 shows a full list of “Transfer In” requests sent or received by a user pharmacy, in accordance with the principles of the present application;

FIG. 6 shows an exemplary “Transfer Out” request form generated by the system of FIG. 1, in accordance with the principles of the present application;

FIG. 7 shows a full list of “Transfer Out” requests sent or received by a user pharmacy, in accordance with the principles of the present application;

FIG. 8 shows an exemplary Transfer Out request acceptance summary, in accordance with the principles of the present application;

FIG. 9 shows an exemplary controlled substance checkpoint alert generated by the system of FIG. 1, in accordance with the principles of the present application;

FIG. 10A shows an exemplary prescription transfer system where a PMS of a receiving pharmacy is fully integrated with a Host System but a PMS of a transferring pharmacy is not;

FIG. 10B shows an exemplary prescription transfer system where a PMS of a transferring pharmacy is fully integrated with a Host System but a PMS of a receiving pharmacy is not;

FIG. 10C shows an exemplary prescription transfer system where a single transferring pharmacy can communicate with multiple receiving pharmacies; and

FIG. 11 shows an exemplary prescription translation module in accordance with the principles of the present application.

### DETAILED DESCRIPTION

Embodiments of the invention provide systems and methods for an electronic platform that enables a compliant communication protocol over a distributed network for the electronic transfer (“E-transfer”) of a prescription between pharmacists at unaffiliated pharmacies, meaning pharmacies operated by different legal entities and using different pharmacy management systems (PMS) that do not share a common prescription database or enterprise network, and therefore cannot natively exchange prescription data securely, accurately and efficiently electronically. According to an embodiment of the invention, the invention is directed to unaffiliated pharmacies that do not share all of the same data because pharmacies that share certain data are excluded from the prescription transfer regulations and may transfer prescriptions electronically without any restrictions. In order to enable the transfer of prescription between unaffiliated pharmacies that do not share the same data, the system may facilitate secure message-based communication between unaffiliated pharmacies, wherein each pharmacy retains control of its own pharmacy management system and database, and wherein prescription data is exchanged through the Host System without granting database-level access between the pharmacies.

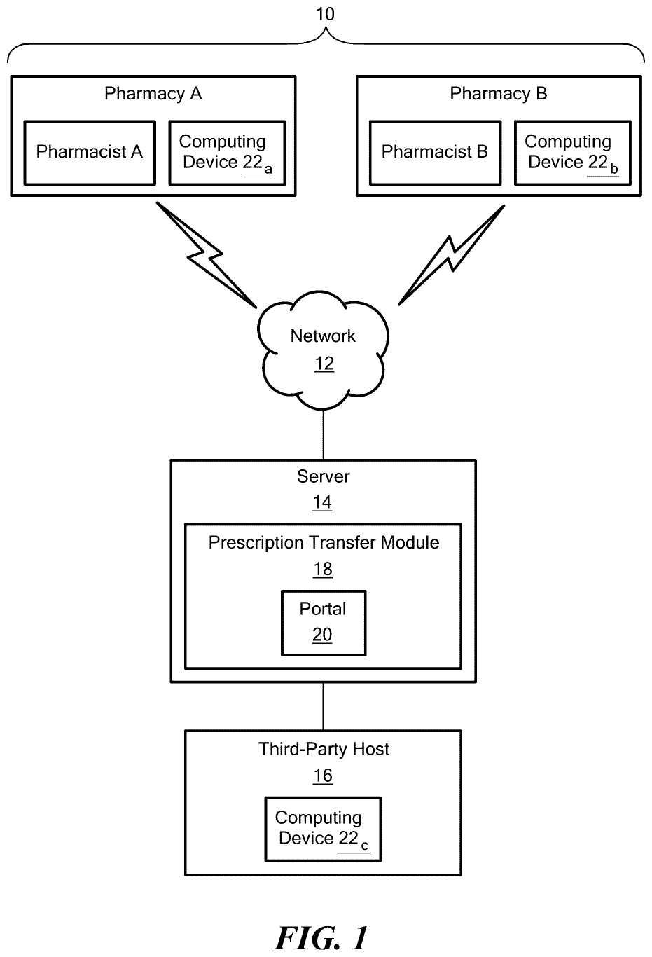

Referring now to the drawings in which like reference designators refer to like elements, there is shown in FIG. 1, a computer-implemented prescription order transfer system 10 that enables a compliant communication protocol between one or more pharmacies, in accordance with principles of the present application. As shown in FIG. 1, the system 10 comprises one or more pharmacies, such as Pharmacy A and Pharmacy B who are members of the system 10. The pharmacies may communicate over network 12 and are each in communication with a host server 14 managed by a third-party host 16. In one or more embodiments, the host sever 14 serves as an intermediary and facilitates communication between each pharmacy through the use of a prescription order transfer module 18 that provides access to a web-based member portal 20. Pharmacy A may include a user or Pharmacist A and a computing device 22a configured to receive input from the Pharmacist A. Similarly, the Pharmacy B may include a second user or Pharmacist B and a computing device 22b configured to receive input from Pharmacist B. The host server 14 may also be accessed by computing device 22. of the host 16.

Continuing to refer to FIG. 1, as described herein the prescription order transfer module 18 is configured to allow each pharmacy to send and/or receive prescription orders and prescription order transfer requests to and from one or more other pharmacies via the portal 20. Thus, it is to be understood that all system functions, operations, and processes necessary to facilitate the prescription order transfer requests described herein, approvals of the prescription order transfer requests, and all other system functions of the portal presented to user pharmacist via the portal 20, are possible through the use of the prescription transfer module 18.

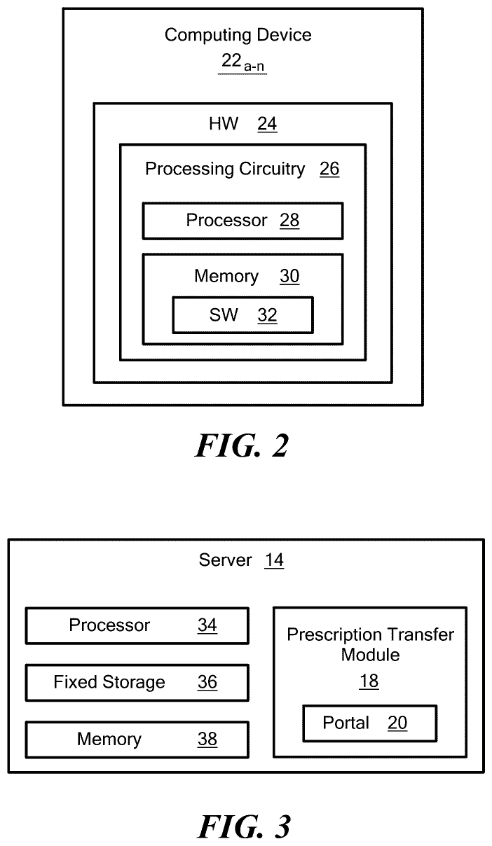

As shown in FIG. 2, computing device 22ₐ₋ₙ further includes hardware and software configured to perform and execute the operations, functions, and processing necessary to implement the system described herein. In some embodiments, the hardware (HW) 24 may include processing circuitry 26 that comprises a processor 28 and a memory 30 in communication with the processor 28. In particular, in addition to or instead of a processor, such as a central processing unit and memory, the processing circuitry 26 may include integrated circuitry for processing and/or control, e.g., one or more processors and/or processor cores and/or FPGAs (Field Programmable Gate Array) and/or ASIC's (Application Specific Integrated Circuitry) adapted or configured to execute instructions. The processor 28 may be configured to access (e.g., write to and/or read from) the memory, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory). Further, memory 30 may be configured as a storage device.

The processing circuitry 26 may be configured to control any of the methods and/or processes described herein and/or to cause such methods and/or processes to be performed by the computing devices 22ₐ₋ₙ described herein. Processor corresponds to one or more processors for performing the computing device functions described herein. In some embodiments, the software 32 may include instructions that, when executed by the processor 28 and/or processing circuitry 26, causes the processor 28 and/or processing circuitry 26 to perform the processes described herein with respect to the computing devices 22ₐ₋ₙ.

The software 32 may be stored internally in, for example, memory 30, or stored in external memory (e.g., database, storage array, network storage device, etc.) and accessible via an external connection. The software 32 may be executable by the processing circuitry 26.

Now referring to FIG. 3, in one or more embodiments the server 14 may include a processor 34, a fixed storage 36, memory 38 in communication with the processor 34 and/or fixed storage 36, and the prescription transfer module 18 that executes the portal 20. In particular, in addition to or instead of a processor, such as a central processing unit and memory, the server 14 may include integrated circuitry for processing and/or control, e.g., one or more processors and/or processor cores and/or FPGAs (Field Programmable Gate Array) and/or ASIC's (Application Specific Integrated Circuitry) adapted or configured to execute instructions. The processor 34 may be configured to access (e.g., write to and/or read from) the memory 38, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory). Further, memory 38 may be configured as a storage device in addition to or in place of fixed storage 36.

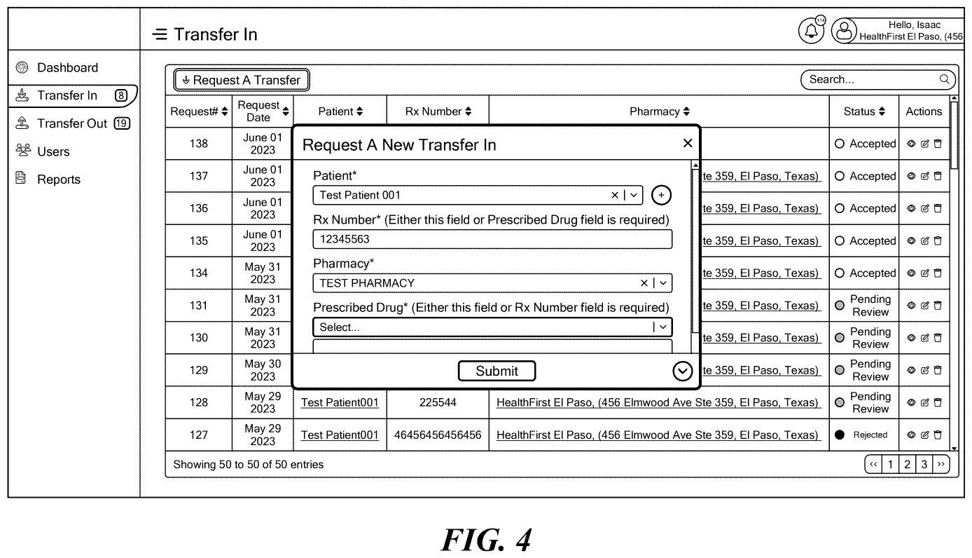

The present system 10 provides a way for pharmacies to quickly and easily communicate with one another when requesting to receive a customer's prescription from another pharmacy, or requesting to send the prescription order to another pharmacy. As an example, in certain situations, upon receiving a prescription order from a customer's prescriber, the customer's existing pharmacy (ex: Pharmacy A) may realize that it cannot fulfill the prescription (e.g., drug out of stock) or the patient may desire the prescription order be transferred to a different pharmacy (ex: Pharmacy B). Pharmacy A may access the portal 20 via their computing device 22ₐ (which is in communication with server 14) to transfer the prescription order (i.e., “Transfer Out” discussed in more detail below) to Pharmacy B that may be able to fulfill the order. Once Pharmacist A identifies a Pharmacy B suitable for receiving the prescription order, it may submit a prescription transfer request through the portal 20 to Pharmacy B, which also has access to the portal 20 via its computing device 22b. Pharmacy B will be notified of the transfer request by a notification alert that will be generated on Pharmacy B's portal account dashboard. It is to be understood that the prescription transfer request may include data and information associated with the patient, details for a particular prescription drug for the patient, and a request for Pharmacy B to accept transfer of the prescription order. Upon being notified that it has received a prescription transfer request, Pharmacist B may navigate to a request page (see FIG. 4) where it may review the details of the prescription transfer request. Upon review, Pharmacist B may then determine if it would like to accept transfer of the prescription order or not. The portal 20 receives user input from Pharmacist B indicating whether the request has been accepted or declined. Upon acceptance or denial, the server 14 generates a notification to alert Pharmacy A of Pharmacy B's decision. If Pharmacy B does accept transfer of the prescription order, the prescription order is immediately available for Pharmacy B to review and process.

As described herein, it is to be understood that the prescription transfer request is accessible and displayed to each user or pharmacist via their respective computing device 22. The prescription transfer request, any subsequent prescription transfers, and any communication between the pharmacies may be conducted over network 12 through the server 14.

Now referring to FIG. 4, a “Transfer In” request form in shown. The system 10 is configured to allow a member pharmacy (i.e., a pharmacy registered to use the portal 20) to “transfer in” and “transfer out” a customer's prescription. For example, the system 10 may generate a fillable web-based form for a user pharmacist to fill out when requesting a “transfer in” of a prescription from another pharmacy. A “Transfer In” is a request by a pharmacy for a prescription order to be transferred to it from another pharmacy. This may apply in situations where the customer is not near the pharmacy and is in need of their prescription. The customer may ask for a nearby pharmacy to request that the prescription order be sent to it so that the customer can have access to their prescription. However, this may also be applicable in a variety of situations such as, for example, a customer simply wants to use a different pharmacy. However, as described herein, a “Transfer In” may also be when a transferring pharmacy receives a request from a receiving pharmacy for the receiving pharmacy to transfer a prescription to the transferring pharmacy. Similarly, a “Transfer Out” request may refer to situations where a pharmacy wishes to send the prescription order out to another pharmacy or has received a request from another pharmacy to transfer a prescription to it.

As shown in FIG. 4, the generated “Transfer In” form may request certain information to be provided by the requesting pharmacist. As a non-limiting example, the requested information may include patient name, pharmacy name, prescription (Rx) number, the prescribed drug and dosage, etc. Once the form has been completed, the system 10 may accept input from the user to send the request to the customer's current pharmacy who is also registered to use the portal 20. In this example, the pharmacy requesting the transfer in is the “receiving pharmacy” and the pharmacy who would be approving the request is the “transferring pharmacy.” Upon receipt of the transfer in request, the transferring pharmacist may review the request and enter input to accept or decline the request. If accepted, the prescription order will be automatically transferred by the system 10 to receiving pharmacy.

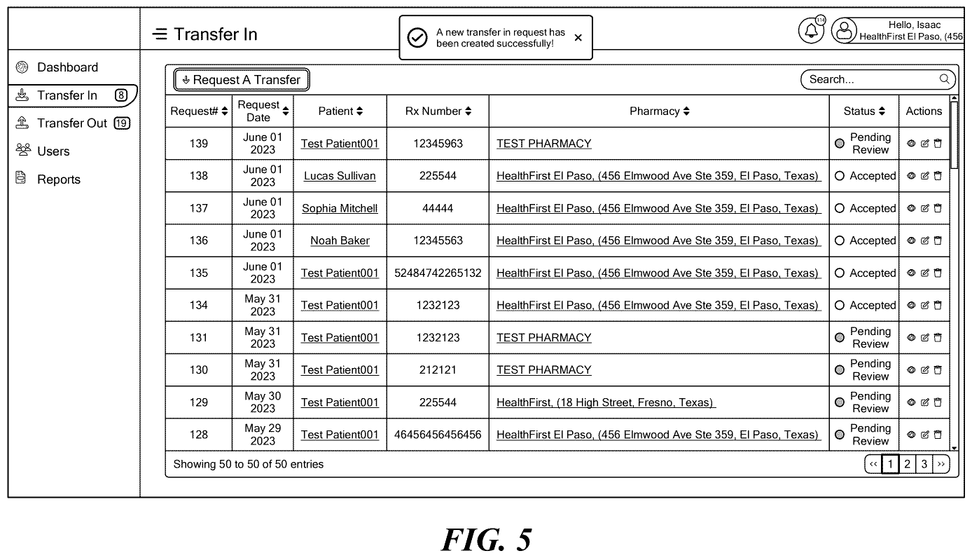

As shown in FIG. 5, the system 10 is also capable of populating a full list of transfer in requests—whether initiated by receiving pharmacy or not. This list shows all of the transfer in request created by the current user pharmacy, as well as the requests received from outside pharmacies. The system 10 shows certain information such as the request number, request date, patient name, Rx number, pharmacy name, status, and action items.

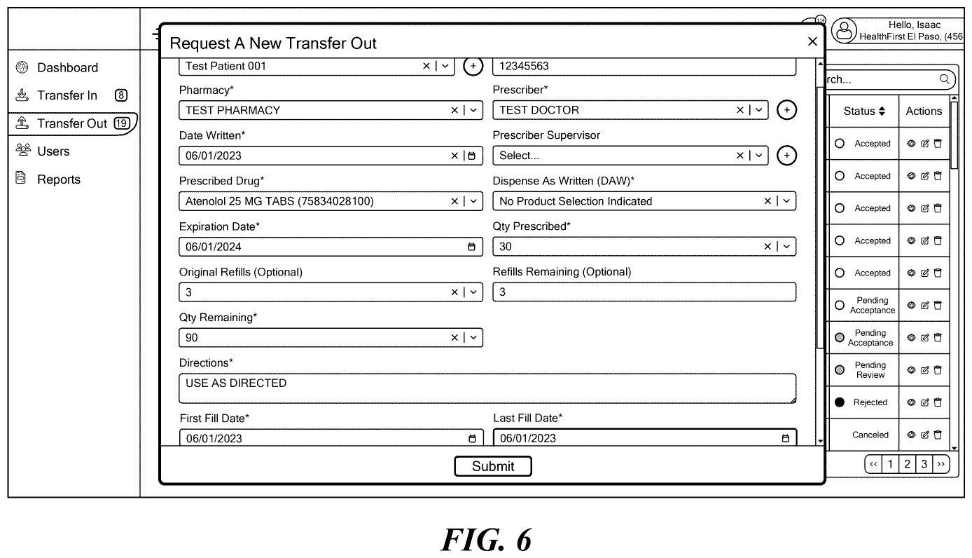

As shown in FIG. 6, the system 10 is also capable of generating “Transfer Out” forms that require transferring pharmacists to enter various information such as, for example, patient name, Rx number, pharmacy name, prescriber name, date written, prescriber supervisor, prescribed drug, dispense as written, expiration date, quantity prescribed, refill amount, refills remaining, quantity remaining, directions, first fill date, and last fill date, etc. Once completed, the transferring pharmacist may enter user input via their computing device 22 to initiate the transfer of the prescription order request to the receiving pharmacy who may then accept or decline the request. If accepted, the receiving pharmacy will be provided with the prescription order and the transferring pharmacy will be alerted via a portal notification of the acceptance. If declined, the transferring pharmacy will be alerted via a notification that the request has been denied. The transferring pharmacy may then generate a new Transfer Out request to send to a different receiving pharmacy.

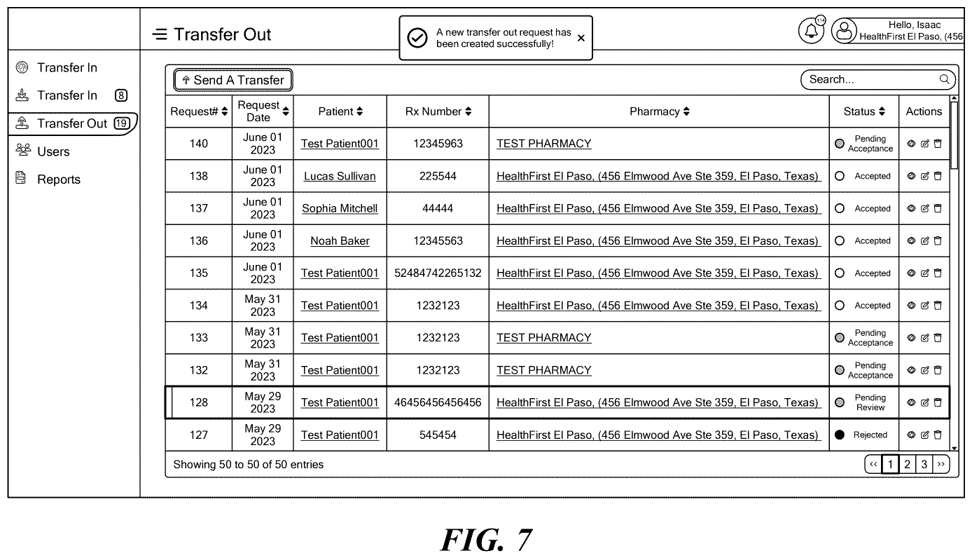

As shown in FIG. 7, the system 10 is also capable of populating a full list of Transfer Out requests—whether initiated by transferring pharmacy or not. This list shows all of the Transfer Out requests created by the current user pharmacy, as well as the requests received from outside pharmacies. The system 10 shows certain information such as the request number, request date, patient name, Rx number, pharmacy name, status, and action items.

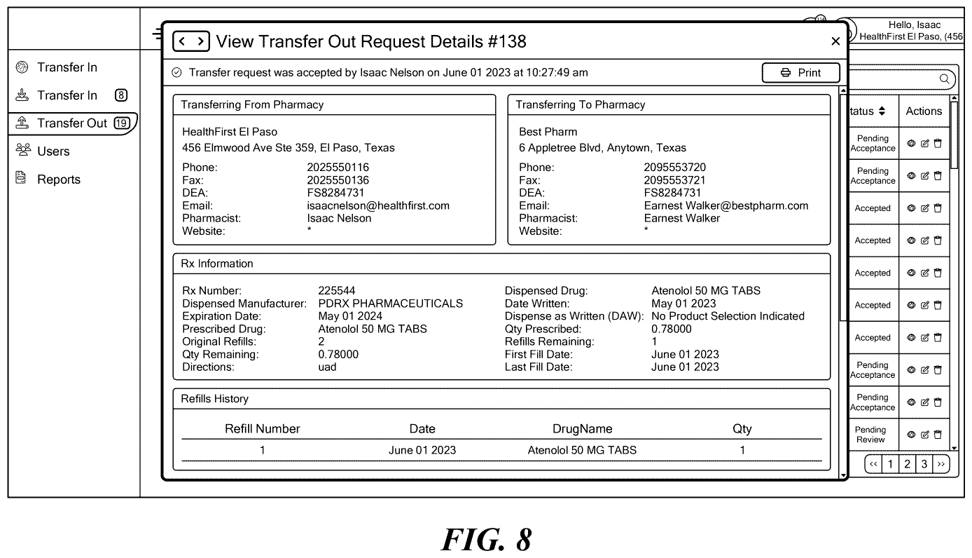

Now referring to FIG. 8, an exemplary Transfer Out request acceptance summary is shown. Upon acceptance of a Transfer Out request, the system 10 is capable of generating a summary that shows the full details associated with the prescription transfer to the other pharmacy. This summary may be downloaded or printed by a pharmacist onto their computing device 22ₐ₋ₙ.

According to one or more embodiments, the pharmacist of each respective pharmacy may manually check their inventory to determine whether a prescribed drug is in stock. This may affect whether the pharmacist chooses to accept or decline a Transfer In request, or initiate a Transfer Out request. However, it is to be understood that in some other embodiments, the system 10 may be configured to access a pharmacies inventory database to automatically determine whether a particular drug is in stock. For example, if a pharmacy wishes to transfer the prescription order out because it does not have it in stock, the system 10 may automatically access and check the inventories of other pharmacies to determine which pharmacies have the drug in stock. Once the system 10 has identified the pharmacies having the drug, the system 10 may alert the transferring pharmacy as to which pharmacies it may transfer the prescription order to.

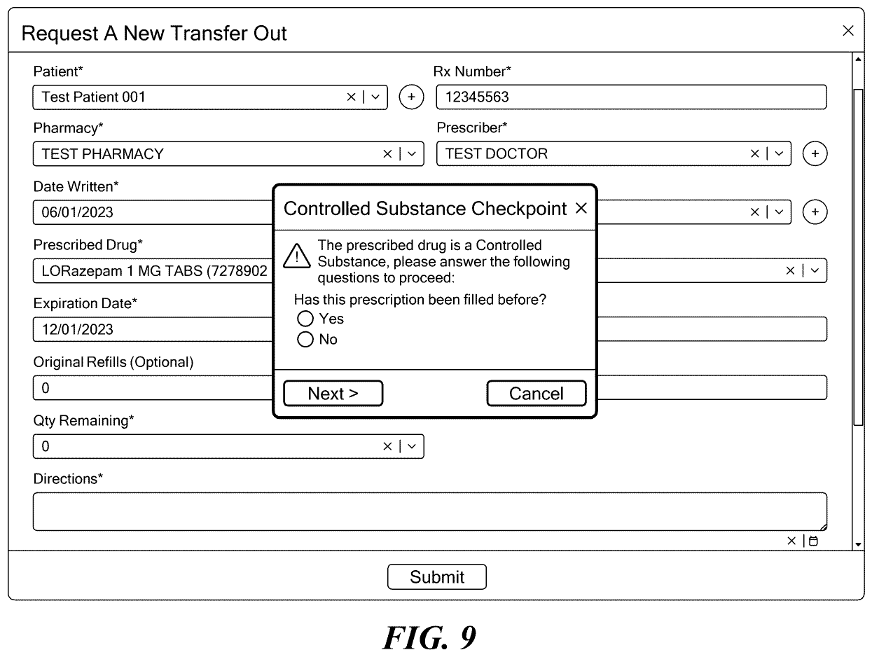

Now referring to FIG. 9, in some embodiments the system 10 is further capable of generating a “Controlled Substance Checkpoint” alert which is presented to a user pharmacist when preparing a Transfer Out request so that the transfer may comply with local or federal regulations. The checkpoint requires the pharmacist to enter additional information when requesting to transfer out controlled substances. Before accepting the Transfer Out Request, the receiving pharmacist may manually check and confirm inventory, and check the Prescription Monitoring Program (PMP) system for control substances abuse, etc. After confirmation by the transferring pharmacists and/or receiving pharmacist, the prescription is then transferred to the receiving pharmacy.

The system 10 may also send a notification at any point during the transfer process to the transferring pharmacy, receiving pharmacy and/or patient regarding the status of the prescription transfer. As an example, the notification may notify the receiving pharmacy that the prescription transfer is waiting for confirmation or to set up secure communication to transfer the prescription. The notification may include a key for the pharmacist of either pharmacy, which may be made up letters and/or numbers or any combination of characters. The key may be used by the notified pharmacist to ensure that a record is maintained in the database regarding the transfer and parts of the transfer in which the notified pharmacist was involved. As described herein, it is to be understood that any and all alerts, notifications, warnings, or messages generated by the system 10 may be displayed to the user pharmacist via a display of their respective computing device 22ₐ₋ₙ. The user pharmacist may interact with any necessary system features through the use of a user interface configured to accept input from the user on the computing device 22.

In some embodiments, the system 10 may also include pharmacy location-based timing thresholds and escalation procedures in order to coordinate the scheduling of pharmacists to transfer prescriptions. The calendars for pharmacists may be stored in order to schedule transfer requests to avoid wasting a pharmacist's time. There may also be escalation procedures, such as urgent notifications, to expedite transfer of the prescription for a customer for any reason. Further, the timing thresholds and escalation procedures may be based on Federal or State Law requirements that require a prescription transfer to occur within a certain time frame. As an example, in order to transfer controlled substances, there is often a time limit-normally, 24 hours but any amount of time is within the scope of this invention. Therefore, the system 10 may provide notifications to ensure the time limit for the transfer of controlled substances is met. The system 10 may also record data as to when certain actions were performed, such as when the request to transfer was entered, when the receiving pharmacy was notified, when the transfer was completed, etc. in order to provide a time trail of what happened, which may be utilized for audit and/or law enforcement purposes and enforcing law.

Further, in some embodiments, the pharmacists and pharmacies may be verified before establishing secure communication and before checking the inventory of the receiving pharmacy in any up-to-date pharmacies database to ensure the pharmacists/pharmacies are verified for transfer. As well, all communications between the databases and pharmacies are secure and encrypted so that the entire transfer is HIPAA compliant. The system 10 may also communicate with controlled substances databases, such as the Drug Enforcement Agency (DEA) or PMP controlled substance databases to display any information regarding the prescription and customer or to update the database after transfer of the prescription. For controlled substance transfers, there may be a questionnaire prompt to either of the pharmacists to verify that the prescription qualifies for a legal transfer as State and Federal law restrict the control substances prescriptions transfer, which will help in reducing controlled substances fraud.

The system 10 may be capable of capturing real-time, time-stamped documentation of communications between pharmacists approving or denying the transfer or any of the steps of the transfer, including any notifications, which will help pharmacists adhere to Federal and State laws that limit the amount of time that is allowed in order for a prescription transfer to be executed. Further, the system 10 may allow a consumer/patient to send their prescription transfer requests securely and directly to the portal in a HIPAA compliant fashion in order to automatically initiate the prescription transfer.

Thus, the system 10 provides an improvement to the transfer of prescriptions through verification of pharmacists, pharmacy location-based timing thresholds and escalation procedures, HIPAA compliant, electronic transfer of information, automated inventory verification, improved pharmacists'and patients'experiences and prescription tracking, and/or optimized controlled substance database review. The system 10 also provides an improvement in the accuracy aspect of transferring of prescription as transfer will not need to be done verbally, which creates a lot of “he said she said” situations as there is no written confirmation in addition to the verbal confirmation, and instead creates a paper trail of requests for transfer, confirmations, notifications, etc. Further, the system 10 will improve patients'lives by getting the patient's medications faster and with less potential for errors.

According to one or more embodiments, a method, system or computer program product to enable a compliant communication protocol over a distributed network for the E-transfer of a prescription between pharmacies includes communicating with a first database, comprising an inventory of a transferring pharmacy, without direct database access or replication between pharmacies, wherein the inventory of the transferring pharmacy comprises a list of prescriptions available at the transferring pharmacy and communicating with a second database comprising an inventory of a receiving pharmacy that is different than the transferring pharmacy, wherein the inventory of the receiving pharmacy comprises a list of prescriptions available at the receiving pharmacy. The method further includes receiving a prescription transfer request from the transferring pharmacy to the receiving pharmacy, wherein the prescription transfer request comprises a prescription and verifying the prescription is available in the inventory of the receiving pharmacy. The method further includes responsive to verifying that the prescription is available in the inventory of the receiving pharmacy, establishing a secure communication between a pharmacist of the transferring pharmacy and a pharmacist of the receiving pharmacy, prompting the pharmacist of the transferring pharmacy to confirm a transfer of the prescription, and responsive to the pharmacist of the transferring pharmacy confirming transfer of the prescription, transferring the prescription to the receiving pharmacy.

According to one or more embodiments, the pharmacist of the transferring pharmacy and the pharmacist of the receiving pharmacy are verified in a NPI or NCPDP database. In another aspect of the embodiment, the transferring of the prescription from the transferring pharmacy to the receiving pharmacy is HIPAA compliant. In yet another aspect of the embodiment, the method further includes communicating with a controlled substance database, displaying information regarding the prescription from the controlled substance database, and automatically updating the controlled substance database after transferring the prescription to the receiving pharmacy. In even yet another aspect of the embodiment, the method further includes storing a calendar for the pharmacist at the transferring pharmacy, storing a calendar for the pharmacist at the receiving pharmacy, and further responsive to verifying that the prescription is available in the inventory of the receiving pharmacy: scheduling a time to establish the secure communication between the pharmacist of the transferring pharmacy and the pharmacist of the receiving pharmacy in the calendar of the pharmacist at the transferring pharmacy and the calendar for the pharmacist at the receiving pharmacy. In another aspect of the embodiment, the scheduling a time to establish the secure communication between the pharmacist of the transferring pharmacy and the pharmacist of the receiving pharmacy is escalated to expedite transfer of the prescription to the receiving pharmacy.

According to one or more embodiments, a data processing system configured to enable a compliant communication protocol over a distributed network for the E-transfer of a prescription between pharmacies includes a host computing system including one or more computers each with memory and at least one processor. The host computing system is in communication with a first database storing an inventory of a transferring pharmacy, wherein the inventory of the transferring pharmacy comprises a list of prescriptions available at the transferring pharmacy and the host computing system is in communication with a second database storing an inventory of a receiving pharmacy that is different than the transferring pharmacy, wherein the inventory of the receiving pharmacy comprises a list of prescriptions available at the receiving pharmacy. The system further includes an application executing in memory of the host computing system and a compliant prescription transfer module coupled to the application. The module includes program code enabled to receive or initiate a prescription transfer request from the transferring pharmacy transferring pharmacy or receiving pharmacy, establishing a secure communication between a pharmacist of the transferring pharmacy and a pharmacist of the receiving pharmacy, prompting the pharmacist of the transferring pharmacy to confirm a transfer of the prescription or prompting the pharmacist of the receiving pharmacy to accept transfer of the prescription; and generating a notification to each pharmacist regarding whether the transfer request has been accepted or declined.

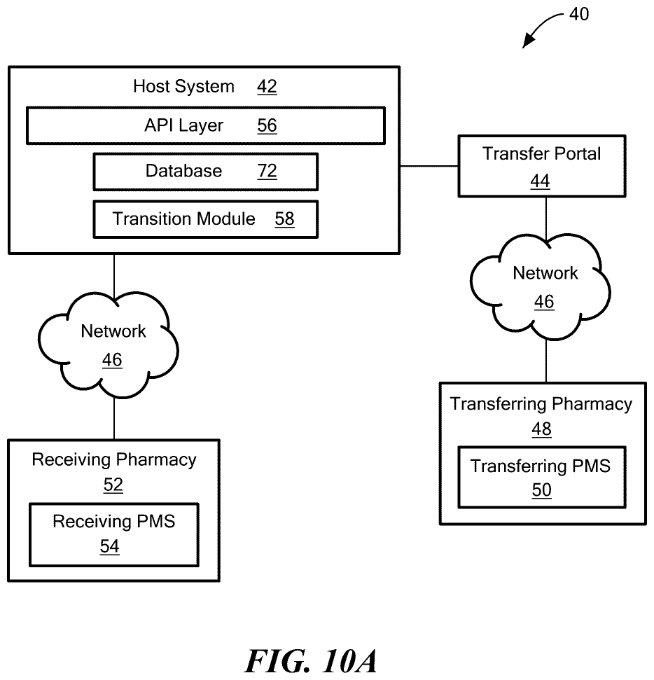

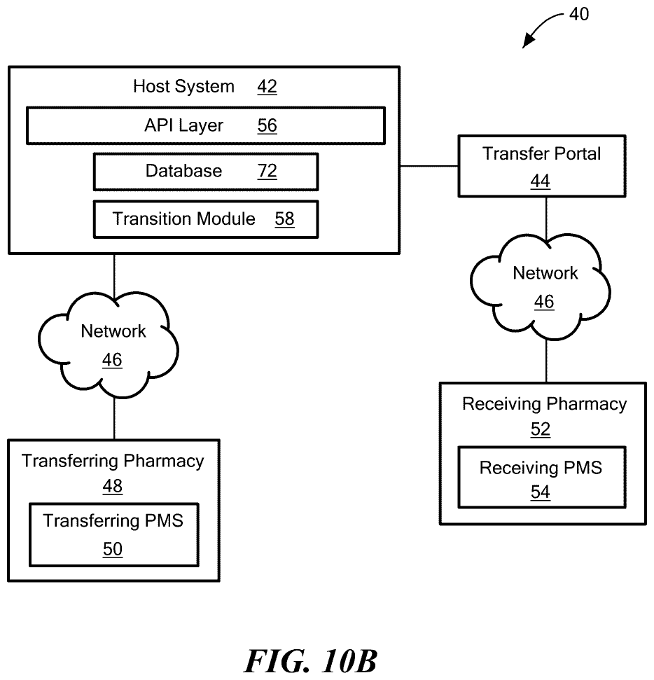

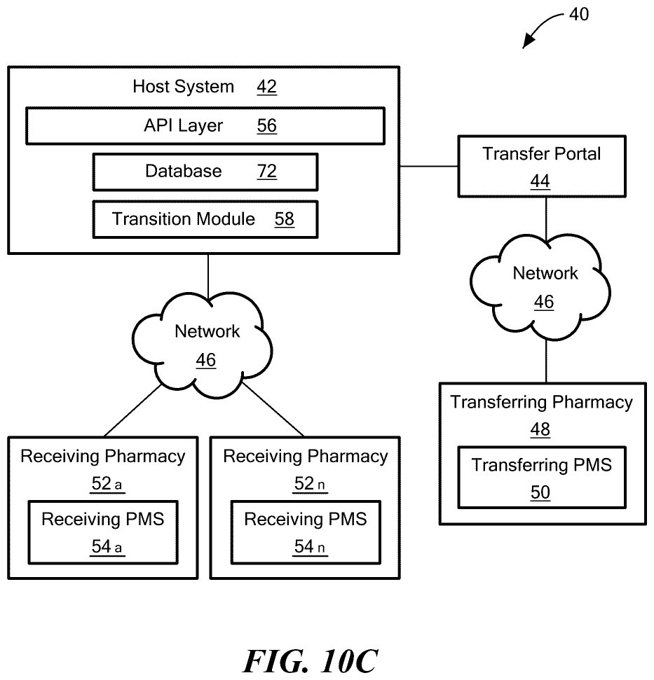

Now referring to FIGS. 10A-10C, an advantageous pharmacy transfer system is configured to allow communication between two or more integrated pharmacies via an API Layer, and to further allow communication between one or more unaffiliated pharmacies-that do not share the same data and operate on separate pharmacy management systems (“PMS”)—and one or more integrated pharmacies via a web-based Transfer Portal that interacts with the API Layer. The present system does not comprise two separate systems but rather comprises a hybrid model that enables communication between two or more pharmacies regardless of whether each pharmacy is integrated with the API Layer. Additionally, the present system comprises a protocol-agnostic translation module that is configured to enable prescription transfers across pharmacy management platforms using different communication formats.

Typically, pharmacies operate in a highly fragmented technology environment, with dozens of incompatible PMS platforms in use. Requiring both pharmacies to be integrated with the same network creates a bottleneck that excludes most real-world transfers. By enabling interoperability when only one side is integrated, the exemplary pharmacy transfer system described herein lowers the technical and financial barriers, ensures that data moves securely and compliantly, and allows unaffiliated pharmacies to exchange prescriptions electronically where previously they could not. The present system removes the requirement that both pharmacies be part of the same software network in order to transfer prescriptions electronically.

As described herein, the term “pharmacy management system” or “PMS system” shall refer to a software platform that runs a pharmacy's daily operations, such as receiving prescriptions, verifying information, billing insurers, managing drug inventory, and documenting dispensing of drugs to fill prescription orders. Each PMS system can be built by a different software vendor and can have its own database structure and internal logic. As described below, the present system 40 provides a Transfer Portal and API Layer that provide a secure digital bridge that allows different software systems to communicate automatically, without human intervention.

As used herein, the terms “unaffiliated pharmacy” or “unaffiliated pharmacies” refers to pharmacies that (i) are operated by different legal entities and/or different corporate groups, (ii) utilize different pharmacy management systems or PMS vendor platforms, and (iii) do not share a common prescription database, enterprise data repository, or closed-network transaction system. The present system connects pharmacies that have no shared software, no shared database, and no shared corporate control.

As shown in FIGS. 10A-10C, the exemplary pharmacy transfer system 40 comprises a Host System 42, a Transfer Portal 44, and one or more networks 46 to facilitate the communication between the Host System 42, Transfer Portal 44, a transferring pharmacy 48 that has a first PMS 50 (the “transfer” or “transferring PMS”) and a receiving pharmacy 52 that has a second PMS 54 (the “receiving PMS”). The Host System 42 comprises an Application Programming Interface (API) Layer 56 that enables the communication between two or more pharmacies that have PMS systems that are integrated with the Host System 42. Integration of the PMS systems with the Host System 42 means that certain information (e.g., patient information, drug information, drug inventory information, prescription transfer elements, etc.) can be securely and efficiently exchanged between the integrated pharmacies via the API Layer 56 using authorized interfaces and message-based communications managed by the Host System 42. However, in many situations it is desired for two or more pharmacies that are not both integrated with the Host System 42 to communicate with one another to process a prescription transfer request. As shown in FIGS. 10A-10C, in such embodiments, the pharmacy transfer system 40 further comprises a web-based Transfer Portal 44 that operates as an intermediary to facilitate communication between two or more pharmacies. In particular, the Transfer Portal 44 enables the communication between two or more pharmacies, even when only one of the participating pharmacies is integrated with the Host System 42. For example, as shown in FIG. 10A, in some embodiments, the transferring PMS 50 is not integrated with the Host System 42 but the receiving pharmacy PMS 54 is. In other embodiments, as shown in FIG. 10B, the receiving PMS 54 is not integrated with the Host System 42 but the transferring pharmacy PMS 50 is.

Continuing to refer to FIGS. 10A-10C, according to one or more embodiments, the system 40 comprises a transferring pharmacy 48 and a receiving pharmacy 52 that are in communication via the Transfer Portal 44 and Host System 42 when either the transferring pharmacy 48 or receiving pharmacy 52 does not use a PMS system that is integrated with the Host System 42. In such situations, the transferring pharmacy 48 and the receiving pharmacy 52 are unaffiliated and operate on separate PMS systems that are not integrated with one another. For example, as shown in FIG. 10A, the transferring pharmacy 48 manages its pharmaceutical inventory and all drug transfers via a transferring PMS 50 that is not integrated with the Host System 42. The receiving pharmacy 52 manages its pharmaceutical inventory and all prescription drug transfer requests via a receiving PMS 50 that is separate from the transferring PMS 48 and is fully integrated with the Host System 42. In order to communicate with the receiving pharmacy 52, the transferring pharmacy 48 must exchange information and data (including information and data related to a prescription transfer request) via the Transfer Portal 44 that is accessible by a user associated with the receiving pharmacy 52 (e.g., the receiving pharmacist or technician) through a web user-interface (“Web UI”). As described herein, it is to be understood that any operations, functions, and/or processes referred to as being performed by the transferring pharmacy 48 are performed by the transferring PMS 50 and any operations, functions, and/or processes referred to as being performed by the receiving pharmacy 52 are performed by the receiving PMS 54.

As shown in FIGS. 10A-10C, the Host System 42 comprises a prescription translation module 58. The translation module 58 operates as middleware between the Transfer Portal/API Layer and the PMS system interfaces of each pharmacy, ensuring that prescription information is validated, normalized, and delivered in a format the receiving PMS 54 can process natively. By performing these protocol-agnostic translations without requiring vendor-specific programming, the pharmacy transfer system 40 enables interoperability across heterogeneous PMS environments, eliminating the need for redundant integrations and significantly improving the efficiency of cross-network prescription transfers.

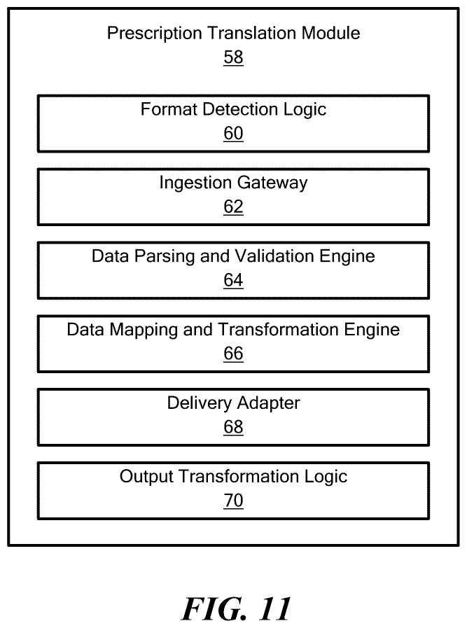

Now referring to FIG. 11, according to one or more embodiments, the prescription translation module 58 comprises (1) format detection logic 60 to identify whether an incoming payload is XML, JSON, HL7-formatted healthcare messages (often encoded in XML or JSON), or another supported communication protocol, (2) a data parsing and validation engine 64 that parses the prescription data, identifying key fields such as patient information, prescriber ID, NDC, dosage, and directions (SIG), (3) a data mapping and transformation engine 66 that converts data elements from the transferring PMS structure into a canonical data model (CDM), and (4) output transformation logic 70 that then maps the CDM elements into the schema and data format requiring by the PMS of the receiving pharmacy 52. As used herein, the term “canonical data model” or “CDM” refers to a universal, standardized way of representing information so that different systems or applications can more easily understand, exchange, and use the exchanged data-even if each system originally stores or formats its data differently.

The data conversion process works as follows:
1. Format Identification: The prescription translation module 58 includes an ingestion gateway 62 for securely receiving incoming prescription data from integrated PMS systems or the Transfer Portal 44. The ingestion gateway 62 can accept data through multiple ingress channels, such as: API endpoints (for integrated PMS systems), secure portal uploads or form submissions (for non-integrated PMS systems); and standardized message protocols (such as HTTPS POST, AS2, or HL7 transport). The ingestion gateway 62 handles authentication, encryption, and message validation before passing the payload forward. It essentially acts as a universal intake point that abstracts how the data arrives-treating all incoming prescriptions as messages to be normalized and processed uniformly. Once data is received, it is immediately handed to the format detection logic 60, which performs lightweight payload introspection to determine the data's structure and format. The format detection logic 60 evaluates: (1) the HTTP Content-Type header (e.g., application/xml, application/json, application/h17); (2) the file extension or MIME type if included; (3) the syntactic markers in the first few bytes (e.g., <?xml for XML or {for JSON); and (4) optional metadata flags within the message envelope, such as version or standard indicators (e.g., “NCPDP SCRIPT 2017071” or “FHIR-R4”). Once the format is identified, the format detection logic 60 routes the message to the corresponding parser (XML parser, JSON, parser, or HL7 handler).
2. Input Parsing: The translation module 58 parses the prescription data, identifying key fields such as patient information, prescriber ID, NDC, dosage, and directions (SIG). The translation module 58 verifies structure and syntax against expected SML and JSON schemas. To perform these processes, the translation module 58 comprises a data parsing and validation engine 64 that is executed by one or more one or more processors and operates immediately after the format detection logic 60 has identified the data type (e.g., XML, JSON). It is responsible for breaking down (“parsing”) the structured data into discrete prescription data fields, verifying that each field complies with expected schemas and standards, and ensuring the prescription is complete and legally transferrable before transformation. For scheme-based parsing, once the format is detected, the system loads the appropriate schema definition-for example, an XML Schema Definition (XSD) for NCPDP SCRIPT, or a JSON Schema for FHIR/JSON models. The data parsing and validation engine 64 of the translation module 58 reads each tag or key-value pair (e.g., <Patient><Name>John Doe</Name></Patient> or {“Patient”: {“Name”: “John Doe”}}) and maps them into discrete internal data structures. During parsing, the system builds a hierarchical object model of the prescription—identifying elements such as patient demographics, prescriber information, drug identifiers (NDC), quantity, directions for use (SIG), and transfer metadata.
3. Canonical Mapping: Each incoming field is mapped into an intermediate canonical model, which acts as a universal template for prescription data. This model defines standardized field names and data types that apply across PMS vendors and communication protocols. The mapping and conversion process is executed by the data mapping and transformation engine 66, which is a subcomponent of the prescription translation module 58. The data mapping and transformation engine 66 operates as a service layer that (1) loads the appropriate inbound and outbound mapping profiles based on the source and destination PMS identified in the integration registry, (2) uses standard transformation technologies such as XSLT for XML, JSONata or JOLT for JSON, or equivalent declarative mappers, (3) applies data validation during conversion to ensure that all required target fields are populated and conform to schema definitions, and (4) generates the fully transformed prescription object and passes it to the delivery adapter 68, which transmits it to the receiving PMS via API or secure channel. This data mapping and transformation engine 66 is specifically configured to transform prescription data between different PMS schemas—even when the systems use incompatible data structures or protocols (e.g., XML vs JSON). It operates after the data has been parsed and validated and before it's sent to the receiving PMS 54.
4. Transformation: Using pre-defined mapping profiles (stored as configuration files or rulesets), the data mapping and transformation engine 66 applies field-level transformations. For example, <Patient><Name>John Doe</Name></Patient>from XML may be converted into {“patient_name”:“John Doe”} in JSON. Unit conversions, field normalization, and data validation (such as NDC formatting) also occur during this step.
5. Output Generation: Once the canonical data is complete, the translation module 58 reconstructs the message according to the receiving PMS system's schema definition. If the receiver expects a JSON payload with a specific nesting pattern, the translation module 58 generates that structure automatically and transmits it securely via the API Layer 56 using output transformation logic 70. According to one or more embodiments, the translation module 58 is configured per recipient from the capability/participant registry: transport (REST API, webhook, SFTP/AS2, message queue), endpoint URLs, auth (mTLS/OAuth2/API key), headers, max payload size, retry/backoff, idempotency keys, and expected acknowledgments. The output generation process occurs after the described mapping and translation processes. The output processes of the translation module 58 does not change data, it delivers it. Common adapter modes (selectable per PMS) include:
REST API client (HTTPS POST/PUT to PMS ingestion endpoint; mTLS/OAuth2).
Webhook invoker (pushes to PMS-registered callback URL).
Secure file delivery (SFTP/AS2 drop to PMS intake folder).
Message broker producer (publishes to PMS queue/topic, e.g., AMQP/Kafka).
Portal pickup (fallback): deposits payload to secure portal inbox when automated ingress is unavailable.

Operational Features: Idempotency (transfer IDs to prevent duplicates), retries with exponential backoff, DLQ/quarantine on failure, ACK/NACK handling, delivery receipts stored in audit log.

The translation process described herein is advantageous because current PMS systems generally require dedicated integrations to handle Extensible Markup Language (XML), the dominant format for legacy prescription standards such as NCPDP SCRIPT. XML is rigid and verbose, using nested “tags” to structure prescription information (e.g., <Patient><Name>John Doe</Name></Patient>). As standards evolve, modern systems are increasingly adopting Javascript Object Notation (JSON), a lightweight, flexible format expressed in key-value pairs (e.g., {“Patient”: {“Name”: “John Doe”} }). JSON is faster to process and easier for developers to work with, but it is not natively compatible with XML-based systems. As a result, PMS vendors are forced to perform separate, redundant integrations: first for XML, and later again for JSON or HL7-FHIR, which leads to inefficiencies, delays, and compliance risks. The present prescription transfer system 40, addresses this problem directly. Instead of requiring each PMS vendor to integrate separately for each protocol, the system described herein allows for transfer requests in a first format to be translated into a second format that is compatible with the PMS system of the receiving pharmacy.

Continuing to refer to FIG. 11, the prescription translation module 58 is configured and/or programmed to translate a prescription transfer request from the transferring PMS system 50 of the transferring pharmacy 48 to a format compatible with the receiving PMS system 54 used by the receiving pharmacy 52. According to one or more embodiments, the format compatible with the receiving PMS system is at least one of XML, JSON, and HL7 formats. When translating the prescription transfer request, the translation module 58 first identifies the data language format of the prescription transfer request and determines what format is compatible with the second PMS system 54. The translation module 58 then maps and converts individual data elements from the format of the transferring PMS system 50 to the format compatible with the receiving PMS system 54. Further, the translation module 58 parses information associated with a pharmaceutical prescription and validates the presence of at least one critical element, such as, for example, patient identifiers, prescriber identifiers, and pharmaceutical drug identifiers. Once the translation process is complete, the prescription transfer request is delivered to the receiving PMS system 54 of the receiving pharmacy 52, which receives the transfer request and presents it to the user at the receiving pharmacy 52. According to one or more embodiments, once the translation process is complete, the prescription transfer request is delivered to the receiving pharmacy 52 through the Host System 42, wherein, if the receiving PMS system 54 is integrated with the Host System 42, the translated prescription data is transmitted via the API Layer 56 and ingested directly into the receiving PMS system 54 for processing within its native workflow, and if the receiving PMS system 54 is not integrated with the Host System 42, the translated prescription data is presented to the receiving pharmacy 52 via the secure web-based interface of the Transfer Portal 44.

On the receiving end, the receiving PMS system 54 of the receiving pharmacy 52 can include an inbound ingestion interface that may comprise one or more of the following:
API Ingestion Endpoint (REST/HTTPS): a documented PMS endpoint that accepts prescription objects; usually performs its own schema validation and enqueues the record into the PMS workflow.
Interface Engine/Listener (health-IT style): a PMS module that listens for HL7/JSON/XML messages, validates, and forwards to the dispensing/verification modules.
Secure Intake Folder/Import Service (SFTP/AS2): a PMS service that polls or is notified of new files, validates schema, and imports into the PMS database.
Message Broker Consumer: a PMS consumer that subscribes to a queue/topic and ingests messages into the PMS.
Downstream PMS path: Ingestion→PMS validation→prescription record creation/update→standard dispensing/billing workflow; because the payload matches the PMS's native schema, the PMS processes it as if created internally.

According to one or more embodiments, the translation process performed by the translation module 58 ensures that prescriptions from the transferring PMS system 50 of the transferring pharmacy 48 appear in the receiving PMS system 54 of the receiving pharmacy 52 as though the two PMS systems were natively integrated with the Host System 42, even when they are not. Unlike existing systems, the present system 40 is a decentralized system that does not require participating pharmacies to be enrolled in or members to a centrally managed network of member pharmacies. The present prescription transfer system 40 bridges the gap between pharmacies that operate independently on different pharmacy management platforms, and without a shared vendor ecosystem.

Although the invention as described herein references a transferring pharmacy and a receiving pharmacy, it is to be understood that the system 40 is adapted to enable the communication between multiple transferring and receiving pharmacies simultaneously and continuously. Additionally, as shown in FIG. 10C, in some embodiments the system 40 can facilitate the communication and transfer of prescription requests from the PMS system of a single transferring pharmacy 48 to the PMS systems of multiple receiving pharmacies 52a-n. In such embodiments, the prescription translation module 58 translates the format of the request from the transferring pharmacy 48 to whichever format is compatible with each of the receiving pharmacies. Additionally, according to some embodiments, prescription transfer requests can be exchanged between two or more pharmacies integrated with the Host System 42 while prescription transfer requests are being managed between two or more pharmacies via the Transfer Portal 44 when at least one of the two pharmacies is not integrated with the Host System 42.

According to one or more embodiments, the PMS systems of the transferring and receiving pharmacies described herein can each comprise one or more modules, such as, for example, a prescription intake module, processing and dispensing module, inventory management module, billing and reimbursement module, compliance and regulatory module, clinical and outcomes-based module, and data exchange module. It is to be understood that each PMS module can communicate and exchange information with the Transfer Portal 44 and/or Host System 42 in order to perform the functions, operations, and system processes described herein.

According to one or more embodiments, the Host System 42 comprises a registry of all participating pharmacies that have PMS systems that are integrated with the Host System 42. The registry is stored in a database 72 of the Host System 42 and holds each pharmacy/PMS record such as, for example, participant ID, PMS vendor, integration mode (API or Portal), capabilities (XML/JSON supported, required auth, endpoints), status flags, and version).

As used herein, the term “integrated” with respect to a pharmacy management system (PMS) means that the PMS has been configured to communicate directly with the Host System 42 through one or more machine-to-machine interfaces, including but not limited to application programming interfaces (APIs), secure message endpoints, or automated data exchange services, such that prescription transfer requests and prescription data may be transmitted, received, and processed automatically without a human reentering data.

A PMS that is “not integrated” is a PMS that does not have such machine-to-machine connectivity with the Host System 42 and therefore interacts with the Host System 42 through the web-based Transfer Portal 44, where prescription information is entered, reviewed, and transmitted by a user associated with the pharmacy via a secure user interface.

According to one or more embodiments, one pharmacy may operate through an integrated PMS while another pharmacy may operate through the Transfer Portal 44, and the Host System 42 is configured to bridge these two modes of participation so that prescription data is exchanged electronically even though only one of the PMS systems is directly integrated.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Computer readable program instructions described herein can be downloaded to respective computing/processing devices from a computer readable storage medium or to an external computer or external storage device via a network. The computer readable program instructions may execute entirely on the user's computer, partly on the user's computer, as a stand-alone software package, partly on the user's computer and partly on a remote computer or entirely on the remote computer or server. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Finally, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

We claim:

1. A method for enabling the interoperability of two or more unaffiliated pharmacies operating on separate pharmacy management (PMS) systems, the method comprising:
receiving, by a Host System, a prescription transfer request submitted by a user associated with a transferring pharmacy through a web-based Transfer Portal, wherein the transferring pharmacy does not use a PMS integrated with the Host System;
identifying, by the Host System, a communication format of the prescription transfer request;
translating, by a prescription translation module of the Host System, the prescription transfer request into a communication format compatible with a receiving PMS system used by a receiving pharmacy that is integrated with the Host System;
transmitting the translated prescription transfer request to the receiving PMS system through an API Layer; and
ingesting, by the receiving PMS system, the translated prescription transfer request into its native prescription processing workflow such that the receiving PMS system processes the prescription transfer request as if the transferring pharmacy were integrated with the Host System.

2. The method of claim 1, wherein the format compatible with the receiving PMS system is at least one of XML, JSON, and HL7-formatted healthcare messages encoded in one of XML or JSON.

3. The method of claim 1, wherein translating the prescription transfer request comprises mapping and converting individual data elements from the format of the first PMS system to the format compatible with the receiving PMS system.

4. The method of claim 3, wherein translating the prescription transfer request further comprises parsing information associated with a pharmaceutical prescription and validating the presence of at least one critical element.

5. The method of claim 4, wherein the at least one critical element comprises a patient identifier, a prescriber identifier, and a pharmaceutical drug identifier.

6. The method of claim 1, wherein the transferring PMS system and receiving PMS system are in communication via an intermediary Host System, the Host System comprising an API Layer and web-based Transfer Portal.

7. The method of claim 6, wherein the Host System further comprises a database storing a registry of one or more member pharmacies that are integrated with Host System.

8. The method of claim 6, wherein the Host System comprises a prescription translation module, the prescription translation module having:
format detection logic;
a data parsing and validation engine;
a data mapping and transformation engine; and
output transformation logic.

9. The method of claim 8, wherein the step of translating the prescription transfer request to a second communication formation comprises:
identifying the first communication format of the prescription transfer request from the transferring PMS system;
parsing data element associated with the prescription transfer request;
converting the data elements into a canonical data model (CDM);
determining which communication formats are compatible with the receiving PMS system; and
mapping the data elements into the second communication format that is compatible with the receiving PMS system.

10. The method of claim 9, wherein the prescription translation module further comprises an ingestion gateway programmed to accept data through one or more ingress channels.

11. A prescription transfer system, comprising:
a Host System comprising:
an API Layer; and
a prescription translation module; and
a web-based Transfer Portal in communication with the Host System;
a transferring PMS system associated with a pharmacy sending a prescription transfer request;
a receiving PMS system associated with a pharmacy receiving the prescription transfer request; and
wherein:
one of the transferring PMS system and receiving PMS system is integrated with the Host System and the other is not;
the Host System translates and delivers the prescription transfer request:
to the receiving PMS via the API Layer when the receiving PMS is integrated; and
for presentation to a user associated with the receiving PMS via the Transfer Portal; and
the Transfer Portal facilitates communication between the transferring PMS system and the receiving PMS system.

12. The system of claim 11, wherein the Host System is configured to:
receive a prescription transfer request from the transferring PMS system; and
identify the communication format of the prescription transfer request.

13. The system of claim 12, wherein the Host System is further configured to:
identify a communication format compatible with the receiving PMS system; and
translate the prescription transfer request to a communication format compatible with the receiving PMS system.

14. The system of claim 12, wherein the prescription translation module comprises:
format detection logic programmed to identify a communication protocol of an incoming information payload;
a data parsing and validation engine;
a data mapping and transformation engine; and
output logic.

15. The system of claim 12, wherein the prescription translation module comprises:
a data parsing and validation engine programmed to parse data elements associated with the prescription transfer request.

16. The system of claim 12, wherein the prescription translation module comprises:
a data mapping and transformation engine programmed to convert data elements from a communication format of the prescription transfer request to a standardized canonical data model format.

17. The system of claim 15, wherein the prescription translation module comprises:
output transformation logic that maps the data elements into the standardized canonical data model format to a format compatible with the receiving PMS system.

18. A prescription transfer system, comprising:
a Host System comprising:
an API Layer; and
a prescription translation module; and
a web-based Transfer Portal in communication with the Host System;
a transferring PMS system that is not integrated with the Host System;
a receiving PMS system integrated with the Host System; and
wherein the Transfer Portal provides a web-based interface for the transferring PMS system to submit prescription transfer requests to the Host System, and the API Layer delivers the translated prescription data for direct ingestion into the receiving PMS system.

19. The system of claim 18, wherein the prescription translation module and API Layer are configured such that the receiving PMS system ingests and processes the prescription transfer request as if the transferring PMS system were natively integrated with the Host System, even though the transferring PMS system is not integrated.
